(19) United States
(12) Patent Application Publication
(10) Pub. No.: US 2024/0207078 A1
(43) Pub. Date: Jun. 27, 2024

(54) Z-SHAPED BRAIDED STENT

(71) Applicant: BEIJING HONGHAI MICROTECH CO., LTD., Beijing (CN)

(72) Inventors: Xuemin Zhang, Beijing (CN); Tao Wang, Beijing (CN); Xunming Ji, Beijing (CN); Jianliang Zheng, Beijing (CN); Jun Lu, Beijing (CN); Yan Yu, Beijing (CN); Ming Li, Beijing (CN); Qiaoyu Zhang, Beijing (CN); Dongdong Chen, Beijing (CN); Yu Zhao, Beijing (CN); Xuzhi Zhang, Beijing (CN); Xianguo Meng, Beijing (CN); Xiaolin Yang, Beijing (CN)

(21) Appl. No.: 18/287,337

(22) PCT Filed: Apr. 12, 2022
(86) PCT No.: PCT/CN22/86371

§ 371 Date: Oct. 18, 2023

(30) Foreign Application Priority Data
Apr. 20, 2021 (CN) 202110428866.2

(51) Int. Cl. A61F 2/91; A61F 2/94

(52) U.S. Cl. CPC A61F 2/91; A61F 2/94

(57) ABSTRACT

A Z-shaped braided stent capable of being implanted into a human organ. The Z-shaped braided stent comprises: a first tubular wire mesh having N braided rings and formed by continuously braiding first braid wires (I) in a Z shape, each braided ring of the first tubular wire mesh having a plurality of first bending points (A) formed by bending the first braid wires (I) and distributed at intervals; and a second tubular wire mesh having N braided rings and formed by continuously braiding second braid wires (II) in a Z shape, each braided ring of the second tubular wire mesh having a plurality of second bending points (B) formed by bending the second braid wires (II) and distributed at intervals. By hooking the first bending points (A) with the second bending points (B), the second tubular wire mesh and the first tubular wire mesh are connected together to form the Z-shaped braided stent. The braided stent can be subjected to axially deformable compression, but axially basically non-deformable stretching.

### TECHNICAL FIELD

The present invention relates to a stent capable of being implanted into a human organ, and more particularly to a braided stent with unique deformation properties.

### BACKGROUND ART

With the development of medical technology, the treatment of various diseases in the human body has been developed into interventional treatment. Among them, the most popular one is a vascular stent, which is basically composed of memory alloy wires braided into a tubular structure with different radii. The stent is deformable in both radial and axial directions to be inserted into the delivery device. After the delivery device enters the specific position of the human body, the stent is released to support the blood vessel to maintain or conduct the flow of blood. Therefore, the deformation of the stent is a common characteristic of the existing stent, and at the same time, the stent is in a pre-set basic form in a released state, then the stent is re-deformed along with the structure of human organs, the re-deformed stent will have a deformation force, which will act on the organs of the human body. In the process of use, at best, the patient will be uncomfortable, or at worst the injury to the organs will occur, resulting in medical accidents. Therefore, due to the diversity of human organs, it is difficult to adapt the existing braided stent to various organ requirements and treatment requirements, so designing stents with specific structures to solve different needs is an urgent problem to be solved.

### SUMMARY OF THE INVENTION

It is an object of the present invention to disclose a stent having a particular braided structure to address a particular desired treatment.

According to a first aspect of the present invention, a Z-shaped braided stent capable of being implanted into a human organ is characterized in that the Z-braided stent is a tubular stent comprising:
a first tubular or cylindrical wire mesh having N braided rings and formed by continuously braiding first braid wires in a Z shape, each braided ring of the first tubular wire mesh having a plurality of first bending points formed by bending the first braid wires and distributed at intervals; and
a second tubular or cylindrical wire mesh having N braided rings and formed by continuously braiding second braid wires in a Z shape, each braided ring of the second tubular wire mesh having a plurality of second bending points formed by bending the second braid wires and distributed at intervals;
wherein, by hooking the first bending points (A) with the second bending points (B), the second tubular wire mesh and the first tubular wire mesh are connected together to form the Z-shaped braided stent;
wherein, by continuously braiding the first braid wires in a Z shape means that the first braid wires are braided into a tubular and continuous first serrated mesh between two adjacent braided rings until being braided into a first tubular wire mesh;
wherein, by continuously braiding the second braid wires in a Z shape means that the second braid wires are braided into a tubular and continuous second serrated mesh between two adjacent braided rings until being braided into a second tubular wire mesh.

Preferably, the bending apex of the first serrated mesh between two adjacent braided rings is the first bending point; the bending apex of the second serrated mesh between two adjacent braided rings is the second bending point.

Preferably, after the first braid wires jump from the first bending point at the tail end of the i^{th} braided ring to the (i+2)^{th} braided ring to start continuous braiding in a Z shape, a plurality of first bending points are formed on the (i+2)^{th} braided ring and the (i+1)^{th} braided ring so as to form a head-end first bending point on the (i+2)^{th} braided ring and a tail-end first bending point on the (i+1)^{th} braided ring; after the second braid wires jump from the second bending point at the tail end of the i^{th} braided ring to the (i+2)^{th} braided ring to start continuous braiding in a Z shape, a plurality of second bending points are formed on the (i+2)^{th} braided ring and the (i+1)^{th} braided ring so as to form a head-end second bending point on the (i+2)^{th} braided ring and a tail-end second bending point on the (i+1)^{th} braided ring.

Preferably, the first and second braided rings and the (N−1)^{th} and Nth braided rings of the first tubular wire mesh and the second tubular wire mesh extend in a circumferential direction parallel to the tubular stent, and the third to (N−2)^{th} braided rings extend in a helical manner at a helical angle α.

Preferably, the helical angle α is 10°-50°.

Preferably, the bending angle β of the first bending point and the second bending point is 30°-60°.

Preferably, the first and second braided rings and the (N−1)^{th} and N^{th} braided rings of the first tubular wire mesh and the second tubular wire mesh extend in a circumferential direction parallel to the tubular stent, and the third to (N−2)^{th} braided rings extend in a helical manner at a helical angle α;
the helical spacing S between the braid wires I and braid wires II from the third braided ring to the N^{th} braided ring is 0.2-10 mm.

Preferably, the first braid wires and/or the second braid wires are made of a degradable material.

Preferably. The Z-shaped braided stent of the present invention further comprises a traction device connecting the N^{th} braided ring of the first tubular wire mesh and the N^{th} braided ring of the second tubular wire mesh, the traction device comprising:
a traction wire braided mesh having one end connected to the N^{th} braided ring of the first tubular wire mesh and the N^{th} braided ring of the second tubular wire mesh; a connecting end connected to the other end of the traction wire braided mesh.

Preferably, the traction wire braided mesh is braided from a plurality of traction wires or is formed by laser etching a metal tube.

Preferably, the connecting ends are offset from the axis of the circular stent.

Preferably, the connecting end is hook-shaped or tubular.

According to another aspect of the present invention, an implementation method for a Z-shaped braided stent capable of being implanted into a human organ, the Z-braided stent being tubular, the method comprises:
forming a first tubular wire mesh with N braided rings by continuously braiding first braid wires in a Z shape, each braided ring of the first tubular wire mesh having a plurality of first bending points formed by bending the first braid wires and distributed at intervals;
after forming a first tubular wire mesh having N braided rings, forming a second tubular wire mesh having N braided rings by continuously braiding second braid wires in a Z shape, each braided ring of the second tubular wire mesh having a plurality of second bending points formed by bending the second braid wires and distributed at intervals;
where, by hooking the first bending points (A) with the second bending points (B), the second tubular wire mesh and the first tubular wire mesh are connected together to form the Z-shaped braided stent;
where, by continuously braiding the first braid wires in a Z shape means that the first braid wires are braided into a tubular and continuous first serrated mesh between two adjacent braided rings until being braided into a first tubular wire mesh;
wherein, continuously braiding the second braid wires in a Z shape means that the second braid wires are braided into a tubular and continuous second serrated mesh between two adjacent braided rings until being braided into a second tubular wire mesh.

The above-mentioned technical solution disclosed in the present invention has a very unique technical feature, the braided stent is axially deformable and compressible, but axially substantially non-deformable and stretchable, the reason being that each bending point is a point where the braid wires I and II hook each other, or is regarded as a limiting point, so as to limit the axial stretching and lengthening, but not limit the axial separation and compression at the bending point; and during compression, the radius of the tubular braided stent is substantially unchanged, and therefore the radius of the entire tubular braided stent is substantially unchanged; another feature of the present invention is that the entire braided stent can be bent and deformed, and after the external force is lost, the deformed braided stent can still maintain the deformed state, that is to say; the deformed braided stent has substantially no internal elastic force for restoring the original state, which is very beneficial for the long-term retention of the braided stent after it is implemented into the human body, so as to avoid applying unnecessary or harmful force to the cavity of the human body.

### BRIEF DESCRIPTION OF THE DRAWINGS

FIG. 1 is a structural schematic view of an embodiment of the present invention;

FIG. 2a and FIG. 2b are schematic views of appearance of an embodiment of the present invention, respectively;

FIG. 3a and FIG. 3b are schematic views of a braiding mold of the present invention, respectively;

FIG. 4 is an expanded view of a braided path of braid wires in FIG. 1;

FIG. 5a and FIG. 5b are schematic views of the braid wires of FIG. 1 on braiding molds, respectively;

FIG. 6 is an expanded view of the braided path of the braid wires in FIG. 1;

FIG. 7 is a partially enlarged structural schematic view of a braided stent;

FIG. 8 is a structural schematic view of a traction device provided at an end portion of the braided stent;

FIG. 9 is a structural schematic view of a connecting end provided at an end portion of the braided stent;

FIG. 10 is a schematic view of a traction device in a braid wire configuration;

FIG. 11a, FIG. 11b and FIG. 11c are schematic views of the appearance of the braided stent as the traction device being a braid wire, respectively;

FIG. 12 is a partially enlarged structural schematic view of the connection between the traction device and the braided stent;

FIG. 13a, FIG. 13b and FIG. 13c are structural schematic views of the traction device as it is a laser etching processing tube, respectively;

FIG. 14 is a partially enlarged structural schematic view of the connection between the traction device and the braided stent;

FIG. 15a, FIG. 15b and FIG. 15c are structural schematic views of hook-shaped connecting ends of end portions of the braided stent, respectively; and

FIG. 16a and FIG. 16b are structural schematic views of tubular connecting ends of end portions of the braided stent, respectively.

### DETAILED DESCRIPTION OF THE INVENTION

A detailed description of the specific embodiments of the present invention is given in conjunction with the accompanying drawings. It should be noted that the detailed description of the specific embodiments is intended to facilitate an understanding of the technical spirit of the present invention, and should not be construed to limit the scope of the claims of the present invention.

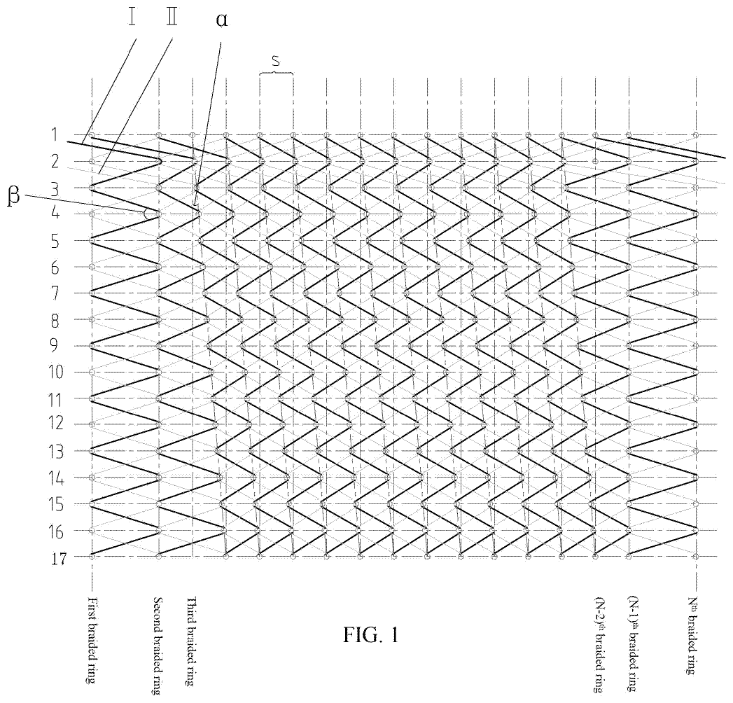

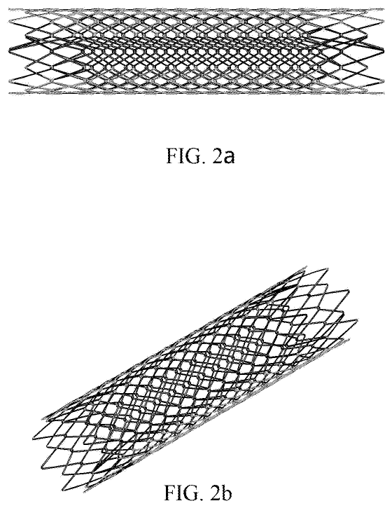

FIG. 1 shows a Z-shaped braided stent capable of being implanted into a human organ in the present invention, the Z-shaped braided stent being tubular as shown in FIGS. 2a and 2b, FIG. 1 is an expanded view of the tubular stent as shown in FIGS. 2a and 2b, wherein columns 1 and 17 are the same.

A Z-shaped braided stent capable of being implanted into a human organ of the present invention comprises:
a first tubular wire mesh (namely, a wire mesh shown by thick solid lines in FIG. 1) having N braided rings and formed by continuously braiding first braid wires I in a Z shape, each braided ring of the first tubular wire mesh having a plurality of first bending points A formed by bending the first braid wires I and distributed at intervals;
and a second tubular wire mesh (namely, a wire mesh shown by thin solid lines in FIG. 1) having N braided rings and formed by continuously braiding second braid wires II in a Z shape, each braided ring of the second tubular wire mesh having a plurality of second bending points B formed by bending the second braid wires II and distributed at intervals;
wherein the Z-shaped braided stent is formed by hooking the first bending point A with the second bending point B (as shown in the figure, the second bending point B hooks the first bending point A from the second braided ring to the (N−1)^{th} braided mesh) so that the second tubular wire mesh and the first tubular wire mesh are connected together;
wherein continuously braiding the first braid wires I in a Z shape means braiding the first braid wires I between two adjacent braided rings into a tubular and continuous first serrated mesh (for example, a serrated mesh shown by a thick solid line between the first braided ring and the second braided ring in FIG. 1) until braiding into a first tubular wire mesh;
here, continuously braiding the second braid wires II in a Z shape means braiding the second braid wires II between two adjacent braided rings into a tubular and continuous second serrated mesh (for example, a serrated mesh shown by a thin solid line between the first braided ring and the second braided ring in FIG. 1) until braiding into a second tubular wire mesh.

As shown in FIG. 1, the bending apex of the first serrated mesh between two adjacent braided rings is a first bending point A; the bending apex of the second serrated mesh between two adjacent braided rings is the second bending point B.

As shown in FIG. 1, a first braid wires I jumps from a first bending point at the tail end of an i^{th} braided ring to an (i+2)^{th} braided ring to start continuous braiding in a Z shape, and a plurality of first bending points are formed on the (i+2)^{th} braided ring and the (i+1)^{th} braided ring so as to form a first bending point at the head end on the (i+2)^{th} braided ring and a first bending point at the tail end on the (i+1)^{th} braided ring; for example, when i is 1, the first braid wires I jumps from the first bending point at the tail end of the first braided ring (the first bending point located in the first row of the first braided ring) to the third braided ring to start continuous braiding in a Z shape, and a plurality of first bending points are formed on the third braided ring and the second braided ring so as to form the first bending point at the head end (the first bending point located in the second row of the third braided ring) on the third braided ring, and the first bending point at the tail end (the first bending point located in the first row of the second braided ring) on the second braided ring.

As shown in FIG. 1, the second braid wires jump from the second bending point at the tail end of the i^{th} braided ring to the (i+2)^{th} braided ring to start continuous braiding in a Z shape, and a plurality of second bending points are formed on the (i+2)^{th} braided ring and the (i+1)^{th} braided ring so as to form a second bending point at the head end on the (i+2)^{th} braided ring and a second bending point at the tail end on the (i+1)^{th} braided ring. For example, when i is 1, the second braid wires II jump from the second bending point at the tail end of the first braided ring (the second bending point located in the second row of the first braided ring) to the third braided ring to start continuous braiding in a Z shape, and a plurality of second bending points are formed on the third braided ring and the second braided ring so as to form a second bending point at the head end (the second bending point located in the third row of the third braided ring) on the third braided ring, and a second bending point at the tail end (the second bending point located in the second row of the second braided ring) on the second braided ring.

As shown in FIG. 1, the first and second braided rings and the (N−1)^{th} and N^{th} braided rings of the first tubular wire mesh and the second tubular wire mesh extend in a circumferential direction parallel to the tubular stent, and the third to (N−2)^{th} braided rings extend in a helical manner at a helical angle α.

As shown in FIG. 1, the braided stent has a helical angle α of 10°-50°. The bending angle β of the first bending point and the second bending point is 30°-60°.

As shown in FIG. 1, the first and second braided rings and the (N-1)^{th} and N^{th} braided rings of the first tubular wire mesh and the second tubular wire mesh extend in a circumferential direction parallel to the tubular stent, and the third to (N−2)^{th} braided rings extend in a helical manner at a helical angle α. The helical spacing S of the braid wires I and II from the third braided ring to the N^{th} braided ring of the braided stent is 0.2-10 mm.

The first braid wires I and/or the second braid wires II of the present invention are composed of the same material or different materials, specifically comprising: combinations of metal wire+metal wire, metal wire+non-metallic wire, non-metallic wire+non-metallic wire. Generally, metal wires are selected from materials such as stainless steel, cobalt-chromium alloy, nickel-titanium alloy, and degradable zinc/magnesium alloy wires, and non-metallic wires are selected from materials such as degradable polylactic acid wires.

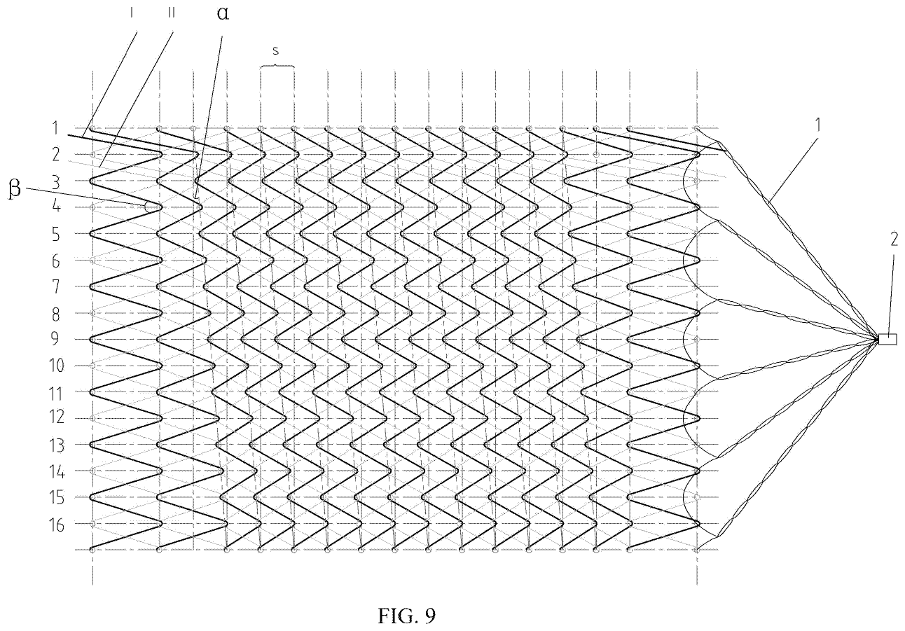

In addition, the present invention further comprises a traction device connecting the N^{th} braided ring of the first tubular wire mesh and the N^{th} braided ring of the second tubular wire mesh, as shown in FIG. 9 to FIG. 16, the traction device comprises: a traction wire braided mesh having one end connected to the N^{th} braided ring of the first tubular wire mesh and the N^{th} braided ring of the second tubular wire mesh; a connecting end connected to the other end of the traction wire braided mesh. The traction wire braided mesh is braided from a plurality of traction wires or is formed by laser etching a metal tube. The connecting ends are offset from the axis of the circular stent. The connecting end is hook-shaped or tubular.

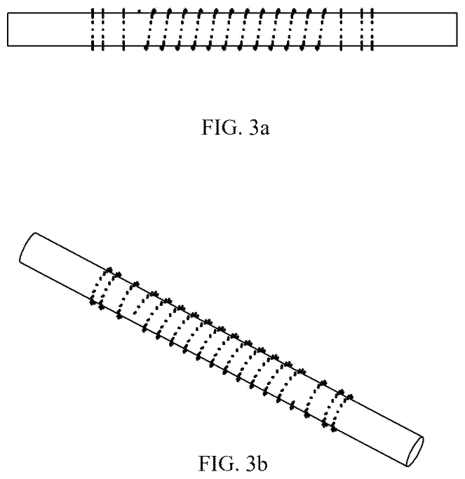

FIGS. 3a and 3b show a mold for braiding a first braid wires I and a second braid wires II according to the present invention, the mold is a tubular object on which a plurality of bumps for braiding are provided.

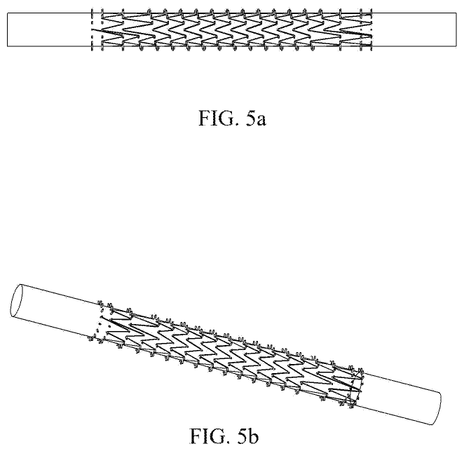

The Z-shaped braided stent capable of being implanted into a human organ of the present invention is made by braiding the first braid wires I or the second braid wires II in a mold according to the marking mode shown in FIGS. 5a and 5b.

An implementation method for a Z-shaped braided stent capable of being implanted into a human organ of the present invention comprises:
a first tubular wire mesh having N braided rings was formed by continuously braiding first braid wires I in a Z shape, each braided ring of the first tubular wire mesh having a plurality of first bending points formed by bending the first braid wires I and distributed at intervals;
after forming a first tubular wire mesh having N braided rings, a second tubular wire mesh having N braided rings formed by continuously braiding second braid wires II in a Z shape, each braided ring of the second tubular wire mesh having a plurality of second bending points formed by bending the second braid wires II and distributed at intervals;
where, by hooking the first bending points (A) with the second bending points (B), the second tubular wire mesh and the first tubular wire mesh are connected together to form the Z-shaped braided stent;
where, by continuously braiding the first braid wires I in a Z shape means that the first braid wires I are braided into a tubular and continuous first serrated mesh between two adjacent braided rings;
where, by continuously braiding the second braid wires II in a Z shape means that the second braid wires II are braided into a tubular and continuous second serrated mesh between two adjacent braided rings.

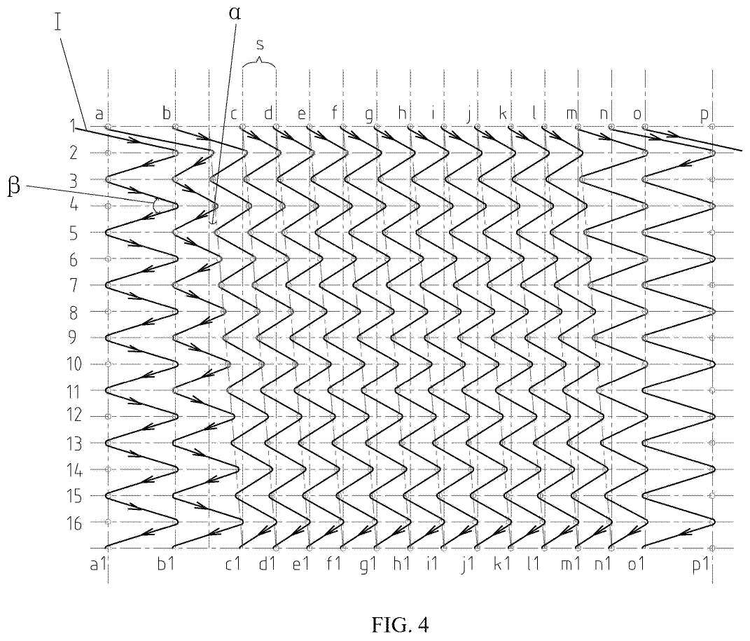

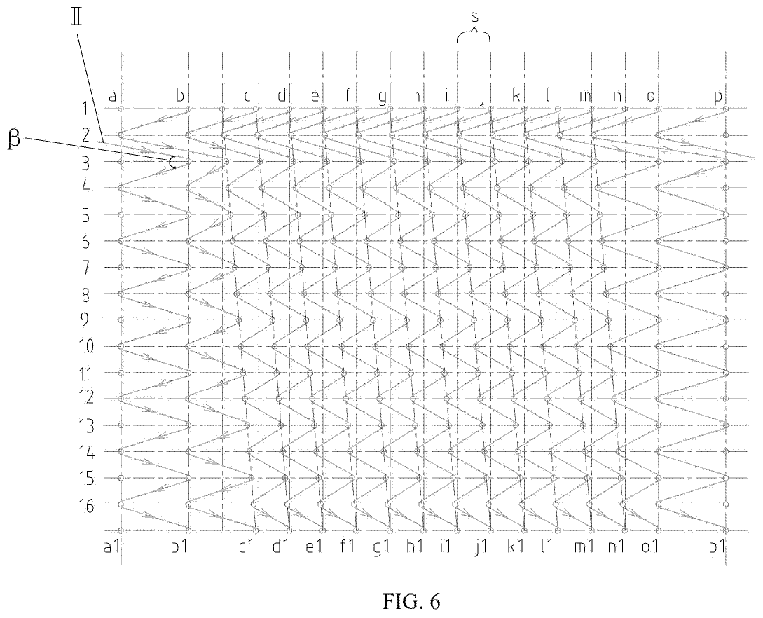

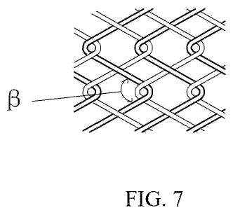

Hereinafter, the present invention will be described in detail with reference to FIGS. 1 to 16. The technical solutions of particular embodiments of the present invention are: the braid wire I of the tubular braided stent is repeatedly continuously bent in a Z shape to form a plurality of bending points, rotates to form a tubular shape, and extends rotationally from one end of the tubular shape to the other end, wherein the first and second bending points are located on the circumference of the same radius, the third to (N−2)^{th} bending points extend to the other end in a helical manner with a helical angle α, and the bending points of the (N−1)^{th} and N^{th} braided rings are located on the circumference of the same radius; the braid wires II is repeatedly continuously bent in a Z shape to form a plurality of bending points, and the braid wires I and II are hooked to each other at the corresponding bending points, and the braid wires II extends from one end of the tubular shape to the other end. The structure of the tubular braided stent according to the above aspect of the present invention is shown in FIG. 1. In order to better understand the three-dimensional structure, the tubular braided stent shown in FIG. 1 is cut in the axial direction and unwound in a plane, and a braid wires I is wound in a braided stent mold (shown in FIG. 3) (shown in FIG. 5). FIG. 4 shows the structure of a flat unwound state of the braid wires I, and FIG. 6 shows the structure of another flat unwound state of the braid wires II; as shown in FIG. 4, a and a₁ are actually the same point, namely, a=a₁, and by the same reasoning, b=b_{1.} and P=P₁; as shown by the arrow in FIG. 4, the braid wires I is repeatedly and continuously bent in a Z shape from point a to point a1, the length of the bent section is the same, namely, returning to point a, and then the repeated bending of the second ring is started as shown by the arrow; the length of the bent section of the braid wires I of the ring is gradually increased, and after point b1, the repeated bending of the third ring is started and repeated to the N^{th} ring, and the length of the bent section of the braid wires I is the same, and the length of the bent section of the braid wires I at the (N+1)^{th} segment is gradually increased The bending sections of the braid wires 1 of the last (N+1)^{th} section have the same length and constitute the structure of the braid wires 1 of the present invention; FIG. 6 shows the structure of another braid wire II in a planar unfolded state, wherein the braid wire II is repeatedly and continuously bent in a Z shape to form a plurality of bending points, and the braid wires I and II are hooked to each other at the corresponding bending points (shown in FIG. 7). As shown in FIGS. 4 and 6, the multiple bending points on the line segments aa₁ and bb₁ are first and second bending points, and the bending points of the first and second circles are located on the circumference of the same radius, while the bending points of the third to (N−2)^{th} circles extend to the other end in a helical manner with a helical angle α, and the bending points of the (N−1)^{th} and N^{th} circles are located on the circumference of the same radius. According to the present invention described above, since the braid wires I and II are hooked to each other and constraint each other at the bending point, when the tubular braided stent is subjected to an axial tensile force, the braided stent does not substantially generate a tensile deformation, and at the same time does not generate a change in the radius; when the braided stent is subjected to an axial compressive force, the hooked structure at the bending point does not restrict the displacement of the braid wires I and II to the centre or the middle position, that is to say, the braid wires I and II at each bending point are disengaged from the hooked state, namely, the braided stent as a whole exhibits a shortened length or a compressed state, and likewise the radius of the braided stent does not change, and the whole braided stent is in a relaxed state; when the braided stent is inserted into the lumen of the human body, the braided stent of the present invention does not change in radius and does not exert force on the wall surface of the lumen of the human body, thereby preventing damage to the lumen of the human body. When the lumen of the human body is in a bent state, the above-mentioned braided stent is bent with the lumen of the human body, the inner bent portion of the bent braided stent is similar to the above-mentioned compressed state, part of the bending points of the braid wires I and II are separated, namely, the inner bent portion is in a relaxed state, while the outer bent portion of the braided stent remains in a hooked state, but at the same time no rebound force after bending is generated; the whole of the whole braided stent is in a relaxed state when the whole is bent, and no clastic force after the whole is bent is generated; therefore, the bent braided stent does not generate an acting force on the lumen of the human body, avoiding damage to the lumen of the human body; or the braided stent of the present invention can be deformed and bent at random, and the bent and deformed state is maintained. The present invention is particularly suitable for curved human lumens, such as curved intestines with a larger diameter of the human body, or cerebral vessels with a thinner brain of the human body, and the braided stent of the present invention greatly improves safety.

In order to prepare a braided stent suitable for human body lumens in various states, the helical angle α of the braided stent is 10°-50°, which can provide a braided stent with different shrinkage rates; the bending angle β of the braid wires I and II of the braided stent is 30°-60°, and the difference in the bending angle β can change the braiding grid density of the braided stent, thereby adjusting the radial support force of the whole braided stent; the bending angle β is preferably 35°-45°; the helical spacing S of the braid wires I and II from the third braided ring to the N^{th} braided ring of the braided stent is 0.2-10 mm, which can adapt to the conditions of braided stents with different diameters; The braided stent is composed of a plurality of braid wires, and the above-mentioned braid wires I and II are both one filament, i.e. two filaments can weave the braided stent of the present invention, but the production efficiency of the preparation process will be reduced, and the braiding efficiency can be greatly improved by a plurality of braid wires, wherein the braid wires of a degradable material account for ⅓-⅔ of the plurality of braid wires, for example, the braided stent is composed of three braid wires according to the above-mentioned braided structure, two are metal alloy wires, and one is a braid wire prepared from a degradable material, or the braided stent is composed of four braid wires, and two are metal alloy wires; two braid wires made of a degradable material, of course, five wires or six wires may constitute a braided stent, and the above-mentioned braid wires of a degradable material may be braided according to the structure of the above-mentioned braid wires; or braiding can be performed after mixing with the metal alloy braid wire; the diameter of the mixed braided metal alloy wire can be reduced without reducing the initial radial supporting force of the braided stent; after entering the human body for a period of time, the degradable braid wire is degraded, which reduces the amount of metal in the lumen of the human body and reduces the rejection reaction of the human body to foreign bodies.

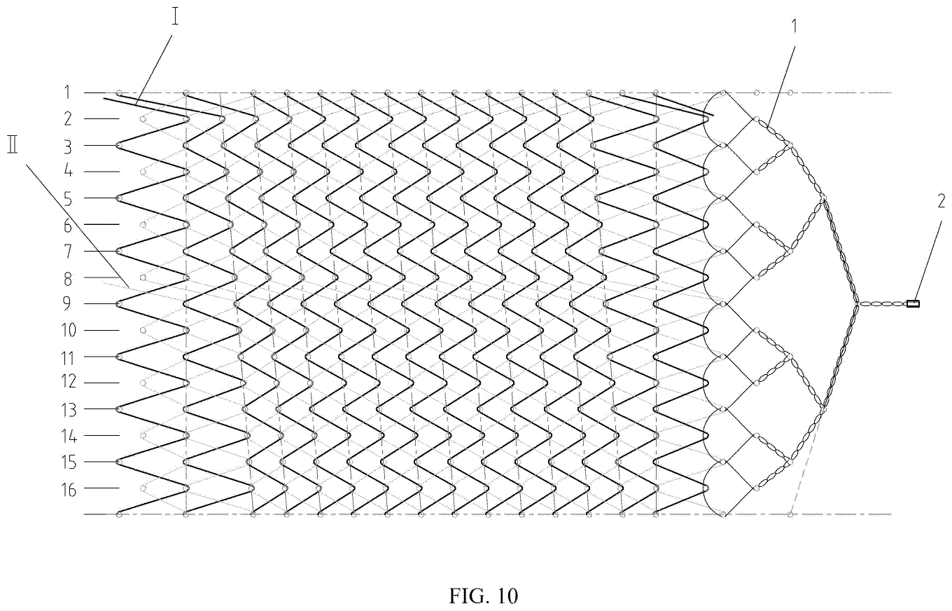

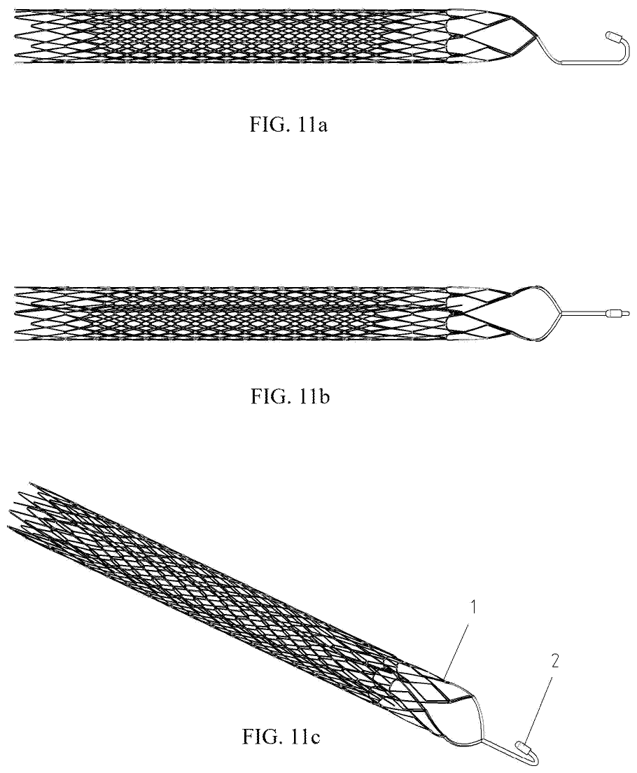

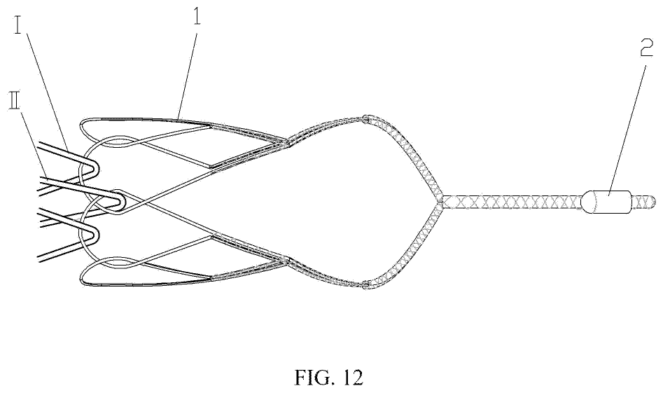

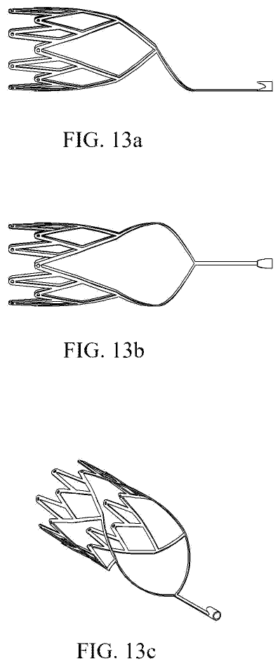

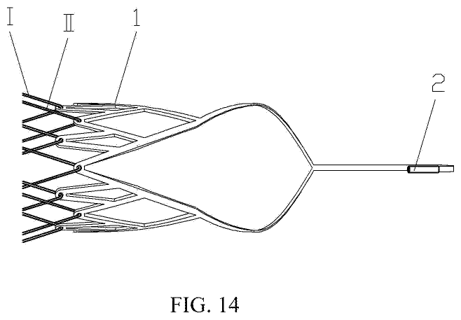

FIG. 10 shows the structure of the traction device which is in a planar unfolded state of the braid wire; FIG. 11 shows a schematic view of the appearance of the traction device after the braided stent of the braid wire is combined; FIG. 12 shows a partially enlarged structural schematic view of the connection between the traction device and the braided stent; FIG. 13 shows a structural schematic view of the traction device which is metal tube formed through laser etching processing; and FIG. 14 shows a partially enlarged structural schematic view of the connection between the traction device and the braided stent.

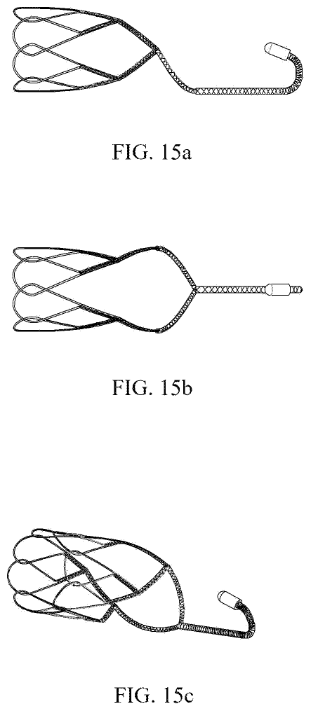

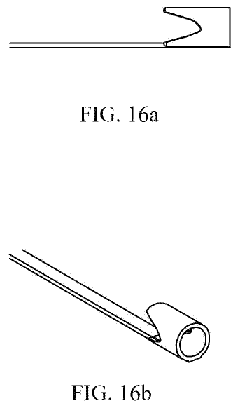

FIG. 15 is a structural schematic view of a hook-shaped connecting end at end portions of the braided stent; FIG. 16 is a structural schematic view of a tubular connecting end at end portions of the braided stent.

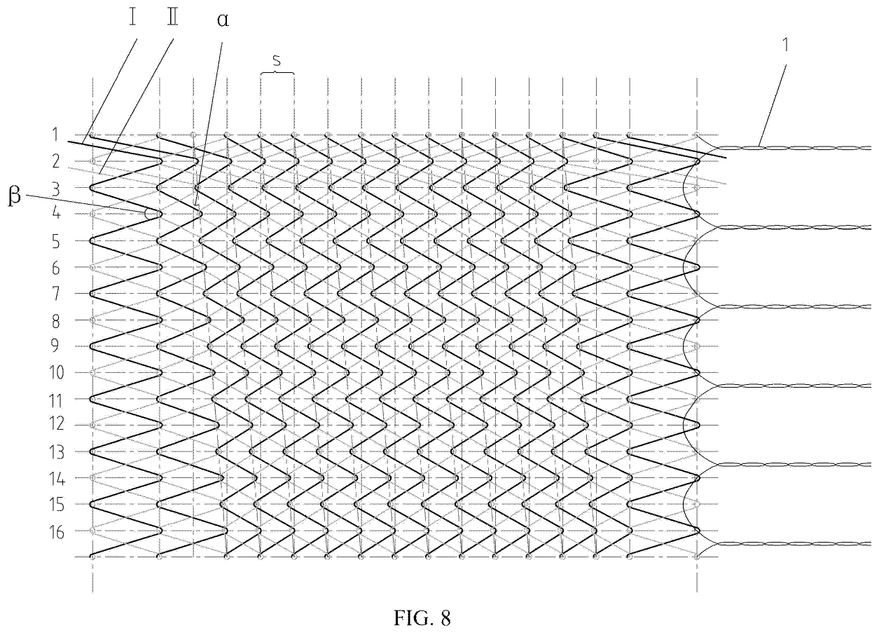

In use, the present invention is placed in a delivery pipe body by forced radial compression and then delivered into a required lumen of a human body, and after release, the braided stent returns to a pre-set normal state to play a supporting role on the lumen; after a period of actual use, the above-mentioned braided stent needs to be taken out from the human body, and therefore one end of the tubular braided stent is provided with a traction device to facilitate the release and recovery of the stent. As shown in FIGS. 8 and 9, the traction device comprises a plurality of traction wires 1 composed of braid wires, wherein the plurality of traction wires I converge and are fixedly connected to a connecting end 2, and the braided stent is recovered and the human body is taken out by connecting to the connecting end 2 via a catching device for inputting into the lumen of the human body, and in order to avoid the influence of the connecting end 2 on the movable object in the lumen of the human body, the above-mentioned connecting end 2 is arranged eccentrically, that is to say, the connecting end 2 is located on the extension line of the braided wall surface of the lumen of the braided stent, which can reduce the flow of blocking liquid and improve the therapeutic effect.

Although the present invention has been described in detail above, the present invention is not limited thereto, and various modifications can be made by those skilled in the art according to the principles of the present invention. Thus, it is intended that the present invention cover the modifications and variations of the present invention provided they come within the scope of the appended claims and their equivalents.

1. A Z-shaped braided stent for being implanted into a human organ, characterized in that the Z-shaped braided stent is a tubular stent, comprising:
a first tubular wire mesh with N braided rings formed by continuously braiding first braid wires in a Z shape, each braided ring of the first tubular wire mesh having a plurality of first bending points formed by bending the first braid wires and distributed at intervals; and
a second tubular wire mesh with N braided rings formed by continuously braiding second braid wires in a Z shape, each braided ring of the second tubular wire mesh having a plurality of second bending points formed by bending the second braid wires and distributed at intervals;
wherein, by hooking the first bending points with the second bending points, the second tubular wire mesh and the first tubular wire mesh are connected together to form the Z-shaped braided stent;
wherein, continuously braiding the first braid wires in a Z shape means that the first braid wires are braided into a tubular and continuous first serrated mesh between two adjacent braided rings of the first tubular wire mesh until being braided into a first tubular wire mesh;
wherein, continuously braiding the second braid wires in a Z shape means that the second braid wires are braided into a tubular and continuous second serrated mesh between two adjacent braided rings of the second tubular wire mesh until being braided into a second tubular wire mesh.

2. The Z-shaped braided stent of claim 1, characterized in that a bending apex of the first serrated mesh between two adjacent braided rings of the first tubular wire mesh is the first bending point; and a bending apex of the second serrated mesh between two adjacent braided rings of the second tubular wire mesh is the second bending point.

3. The Z-shaped braided stent of claim 1, characterized in that after the first braid wires jump from the first bending point at the tail-end of the i^{th} braided ring of the first tubular wire mesh to the (i+2)^{th} braided ring of the first tubular wire mesh to start continuous braiding in a Z shape, a plurality of first bending points are formed on the (i+2)^{th} braided ring of the first tubular wire mesh and the (i+1)^{th} braided ring of the first tubular wire mesh so as to form a head-end first bending point on the (i+2)^{th} braided ring of the first tubular wire mesh and a tail-end first bending point on the (i+1)^{th} braided ring; after the second braid wires jump from the second bending point at the tail end of the i^{th} braided ring of the second tubular wire mesh to the (i+2)^{th} braided ring of the second tubular wire mesh to start continuous braiding in a Z shape, a plurality of second bending points are formed on the (i+2)^{th} braided ring of the second tubular wire mesh and the (i+1)^{th} braided ring of the second tubular wire mesh so as to form a head-end second bending point on the (i+2)^{th} braided ring of the second tubular wire mesh and a tail-end second bending point on the (i+1)^{th} braided ring of the second tubular wire mesh;
wherein i=1, 2 . . . N.

4. The Z-shaped braided stent of claim 1, characterized in that the first braided ring and the second braided ring and the (N−1)^{th} and N^{th} braided rings of the first tubular wire mesh and the second tubular wire mesh extend in a circumferential direction parallel to the tubular stent, and the third to (N−2)^{th} braided rings of the first tubular wire mesh and the second tubular wire mesh extend in a helical manner at a helical angle α.

5. The Z-shaped braided stent of claim 4, characterized in that the helical angle α is 10°-50°.

6. The Z-shaped braided stent of claim 1, characterized in that the first bending point and the second bending point have a bending angle β of 30°-60°.

7. The Z-shaped braided stent of claim 1, characterized in that the first braided ring and the second braided ring and the (N−1)^{th} and N^{th} braided rings of the first tubular wire mesh and the second tubular wire mesh extend in a circumferential direction parallel to the tubular stent, and the third to (N−2)^{th} braided rings of the first tubular wire mesh and the second tubular wire mesh extend in a helical manner at a helical angle α;
the helical spacing S between first braid wires and second braid wires from the third braided ring to the N^{th} braided ring of the first tubular wire mesh and the second tubular wire mesh is 0.2-10 mm.

8. The Z-shaped braided stent of claim 1, characterized in that the first braid wires and/or second braid wires are composed of the same material or different materials, in particular comprising: combinations of metal wire+metal wire, metal wire+non-metallic wire, non-metallic wire+non-metallic wire;
wherein, the metal wires are selected from materials such as stainless steel, cobalt-chromium alloy, nickel-titanium alloy, and degradable zinc/magnesium alloy wires, and the non-metallic wires are selected from materials such as degradable polylactic acid wires.

9. The Z-shaped braided stent of claim 1, characterized by further comprising a traction device connecting the N^{th} braided ring of the first tubular wire mesh and the N^{th} braided ring of the second tubular wire mesh, the traction device comprising:
a traction wire braided mesh having one end connected to the N^{th} braided ring of the first tubular wire mesh and the N^{th} braided ring of the second tubular wire mesh; a connecting end connected to the other end of the traction wire braided mesh.

10. The Z-shaped braided stent of claim 9, characterized in that the traction wire braided mesh is braided from a plurality of traction wires or is formed by laser etching a metal tube.

11. The Z-shaped braided stent of claim 10, characterized in that the connecting ends are offset from the axis of the tubular stent.

12. The Z-shaped braided stent of claim 10, characterized in that the connecting ends are hook-shaped or tubular.

13. An implementation method for a Z-shaped braided stent for implanting into a human organ, the Z-shaped braided stent being a tubular stent, characterized in that the method comprises:
forming a first tubular wire mesh with N braided rings by continuously braiding first braid wires in a Z shape, each braided ring of the first tubular wire mesh having a plurality of first bending points formed by bending the first braid wires and distributed at intervals;
after forming a first tubular wire mesh with N braided rings, forming a second tubular wire mesh having N braided rings by continuously braiding second braid wires in a Z shape, each braided ring of the second tubular wire mesh having a plurality of second bending points formed by bending the second braid wires and distributed at intervals;
wherein, by hooking the first bending points with the second bending points, the second tubular wire mesh and the first tubular wire mesh are connected together to form the Z-shaped braided stent;
wherein, continuously braiding the first braid wires in a Z shape means that the first braid wires are braided into a tubular and continuous first serrated mesh between two adjacent braided rings of the first tubular wire mesh until being braided into a first tubular wire mesh;
wherein, continuously braiding the second braid wires in a Z shape means that the second braid wires are braided into a tubular and continuous second serrated mesh between two adjacent braided rings of the second tubular wire mesh until being braided into a second tubular wire mesh.

14. The method of claim 13, characterized in that a bending apex of the first serrated mesh between two adjacent braided rings of the first tubular wire mesh is the first bending point; and a bending apex of the second serrated mesh between two adjacent braided rings of the second tubular wire mesh is the second bending point.

15. The method of claim 13, characterized in that after the first braid wires jump from the first bending point at the tail end of the i^{th} braided ring of the first tubular wire mesh to the (i+2)^{th} braided ring of the first tubular wire mesh to start continuous braiding in a Z shape, a plurality of first bending points are formed on the (i+2)^{th} braided ring of the first tubular wire mesh and the (i+1)^{th} braided ring of the first tubular wire mesh until forming a head-end first bending point on the (i+2)^{th} braided ring of the first tubular wire mesh and a tail-end first bending point on the (i+1)^{th} braided ring of the first tubular wire mesh; after the second braid wires jump from the second bending point at the tail end of the i^{th} braided ring of the second tubular wire mesh to the (i+2)^{th} braided ring of the second tubular wire mesh to start continuous braiding in a Z shape, a plurality of second bending points are formed on the (i+2)^{th} braided ring of the second tubular wire mesh and the (i+1)^{th} braided ring of the second tubular wire mesh until forming a head-end second bending point on the (i+2)^{th} braided ring of the second tubular wire mesh and a tail-end second bending point on the (i+1)^{th} braided ring of the second tubular wire mesh;
wherein i=1, 2 . . . N.

16. The method of claim 13, characterized in that the first braided ring and the second braided ring, and the (N−1)^{th} braided ring and N^{th} braided rings of the first tubular wire mesh and the second tubular wire mesh extend in a circumferential direction parallel to the tubular stent, and the third to (N−2)^{th} braided rings of the first tubular wire mesh and the second tubular wire mesh extend in a helical manner at a helical angle α.

17. The method of claim 16, characterized in that the helical angle α is 10°-50°.

18. The method of claim 13, characterized in that the first bending point and the second bending point have a bending angle β of 30°-60°.

19. The method of claim 13, characterized in that the first and second braided rings and the (N−1)^{th} and N^{th} braided rings of the first tubular wire mesh and the second tubular wire mesh extend in a circumferential direction parallel to the tubular stent, and the third to (N−2)^{th} braided rings of the first tubular wire mesh and the second tubular wire mesh extend in a helical manner at a helical angle α;
the helical spacing S between first braid wires and second braid wires from the third braided ring to the N^{th} braided ring of the first tubular wire mesh and the second tubular wire mesh is 0.2-10 mm.

20. The method of claim 13, characterized in that a traction device connecting the N^{th} braided ring of the first tubular wire mesh and the N^{th} braided ring of the second tubular wire mesh, the traction device comprises:
a traction wire braided mesh having one end connected to the N^{th} braided ring of the first tubular wire mesh and the N^{th} braided ring of the second tubular wire mesh;
a connecting end connected to the other end of the traction wire braided mesh.
